(19) United States
(12) Patent Application Publication
(10) Pub. No.: US 2016/0361005 A1
(43) Pub. Date: Dec. 15, 2016

(54) BLOOD OXYGEN AND ELECTROCARDIOGRAPHY MEASURING APPARATUS

(71) Applicant: Leadtek Research Inc., New Taipei City (TW)

(72) Inventor: Cheng-Jun Chuang, New Taipei City (TW)

(21) Appl. No.: 15/052,830

(22) Filed: Feb. 24, 2016

(30) Foreign Application Priority Data
Jun. 12, 2015 (TW) 104209464

(51) Int. Cl. A61B 5/1455; A61B 5/00; A61B 5/024; A61B 5/0205

(52) U.S. Cl. CPC A61B 5/14552; A61B 5/0205; A61B 5/6826; A61B 5/02416

(57) ABSTRACT

A blood oxygen and electrocardiography measuring apparatus, including a first clipping component, a second clipping component, a heart rate sensing pad, and a blood oxygen sensing component, is provided. The second clipping component and the first clipping component are pivoted with each other. The heart rate sensing pad is disposed on the second clipping component and located between the first clipping component and the second clipping component. The blood oxygen sensing component is disposed on the second clipping component and located between the second clipping component and the heart rate sensing pad. The blood oxygen sensing component is exposed from the heart rate sensing pad.

### CROSS-REFERENCE TO RELATED APPLICATION

This application claims the priority benefit of Taiwan application serial no. 104209464, filed on Jun. 12, 2015. The entirety of each of the above-mentioned patent applications is hereby incorporated by reference herein and made a part of this specification.

### BACKGROUND OF THE INVENTION

Field of the Invention

The invention relates to a blood oxygen and electrocardiography measuring apparatus and particularly relates to a finger clip type blood oxygen and electrocardiography measuring apparatus.

Description of Related Art

There is an increasing awareness of the importance of health care. To meet the surging demand, a wide range of measuring apparatuses is available on the market. With use of such measuring apparatuses, the users are able to check their current physical conditions, such as blood oxygen concentration, heart rate, and other body-related data, instantly. Common non-invasive measuring apparatuses, e.g. finger clip type ECG (electrocardiography) probe, strap type ECG probe, finger clip type blood oxygen probe, and strap type blood oxygen probe, can only measure either of the user's heart rate and blood oxygen concentration. That is, in order to measure blood oxygen concentration and heart rate, the user will need to wear both the finger clip type ECG probe (or strap type ECG probe) and the finger clip type blood oxygen probe (or strap type blood oxygen probe).

Thus, how to integrate the functions of measurement of blood oxygen concentration and heart rate in one non-invasive measuring apparatus so as to be more convenient to use is an issue that needs to be addressed.

### SUMMARY OF THE INVENTION

The invention provides a blood oxygen and electrocardiography measuring apparatus that is convenient to use.

The invention provides a blood oxygen and electrocardiography measuring apparatus, including a first clipping component, a second clipping component, a heart rate sensing pad, and a blood oxygen sensing component. The second clipping component and the first clipping component are pivoted with each other. The heart rate sensing pad is disposed on the second clipping component and located between the first clipping component and the second clipping component. The blood oxygen sensing component is disposed on the second clipping component and located between the second clipping component and the heart rate sensing pad. The blood oxygen sensing component is exposed from the heart rate sensing pad.

In an embodiment of the invention, the blood oxygen sensing component is an optical transceiver.

In an embodiment of the invention, the heart rate sensing pad includes a body portion, an extension portion connected with the body portion, and an opening located on the body portion. The blood oxygen sensing component is electrically connected with the extension portion. The opening exposes the blood oxygen sensing component.

In an embodiment of the invention, the blood oxygen and electrocardiography measuring apparatus further includes a connection terminal. The connection terminal is disposed in contact with the extension portion to be electrically connected with the heart rate sensing pad.

In an embodiment of the invention, the blood oxygen and electrocardiography measuring apparatus further includes at least one elastic member. The elastic member connects the first clipping component and the second clipping component.

The invention provides another blood oxygen and electrocardiography measuring apparatus, including a first clipping component, a second clipping component, a finger pad, a heart rate sensing pad, and a blood oxygen sensing module. The second clipping component and the first clipping component are pivoted with each other. The finger pad is disposed on the first clipping component and located between the first clipping component and the second clipping component. The heart rate sensing pad is disposed on the second clipping component and located between the first clipping component and the second clipping component. The blood oxygen sensing module is disposed on the second clipping component and located between the second clipping component and the heart rate sensing pad. The blood oxygen sensing module includes an optical transmitter and an optical receiver. The optical transmitter is disposed on the first clipping component and located between the first clipping component and the finger pad. The finger pad exposes the optical transmitter. The optical receiver is disposed on the second clipping component corresponding to the optical transmitter and located between the second clipping component and the heart rate sensing pad. The heart rate sensing pad exposes the optical receiver.

In an embodiment of the invention, the heart rate sensing pad includes a body portion, an extension portion connected with the body portion, and an opening located on the body portion. The optical receiver is electrically connected with the extension portion. The opening exposes the optical receiver.

In an embodiment of the invention, the blood oxygen and electrocardiography measuring apparatus further includes a circuit board. The circuit board is disposed between the first clipping component and the finger pad. The optical transmitter is electrically connected with the circuit board.

Based on the above, the blood oxygen and electrocardiography measuring apparatus of the invention clips the user's finger between the first clipping component and the second clipping component and measures the blood oxygen concentration and/or heart rate of the user by the heart rate sensing pad and/or the blood oxygen sensing component (or blood oxygen sensing module), thereby improving the convenience.

To make the aforementioned and other features and advantages of the invention more comprehensible, several embodiments accompanied with drawings are described in detail as follows.

### BRIEF DESCRIPTION OF THE DRAWINGS

The accompanying drawings are included to provide a further understanding of the invention, and are incorporated in and constitute a part of this specification. The drawings illustrate exemplary embodiments of the invention and, together with the description, serve to explain the principles of the invention.

FIG. 1A is a cross-sectional schematic view depicting the blood oxygen and electrocardiography measuring apparatus in a closed state according to an embodiment of the invention.

FIG. 1B is a cross-sectional schematic view of the blood oxygen and electrocardiography measuring apparatus of FIG. 1A that has been converted to an expanded state.

FIG. 2 is a cross-sectional schematic view depicting the blood oxygen and electrocardiography measuring apparatus in the expanded state according to another embodiment of the invention.

### DESCRIPTION OF THE EMBODIMENTS

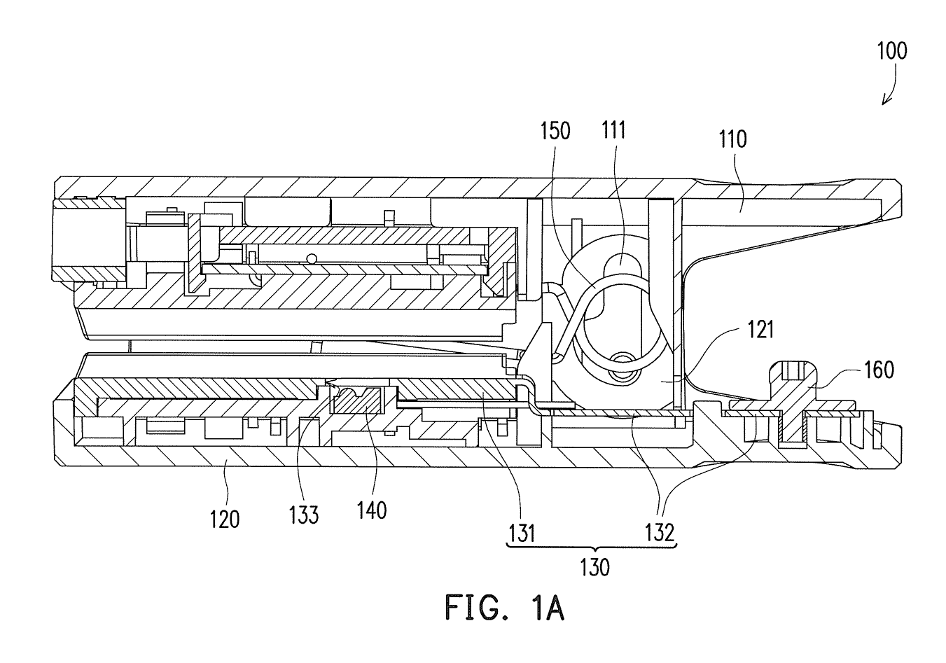

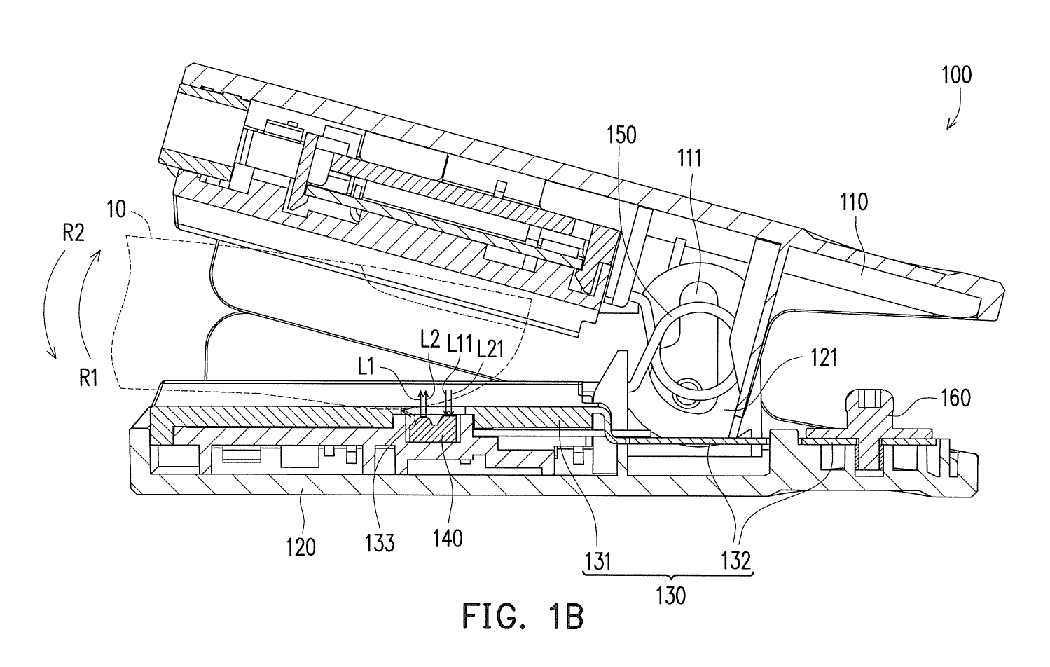

FIG. 1A is a cross-sectional schematic view depicting a blood oxygen and electrocardiography measuring apparatus in a closed state according to an embodiment of the invention. FIG. 1B is a cross-sectional schematic view of the blood oxygen and electrocardiography measuring apparatus of FIG. 1A that has been converted to an expanded state. With reference to FIG. 1A and FIG. 1B, in this embodiment, a blood oxygen and electrocardiography measuring apparatus 100 is a finger clip type blood oxygen and electrocardiography measuring probe, for example, which simultaneously measures the heart rate and blood oxygen concentration of the user or measures one of the heart rate and blood oxygen concentration by clipping a finger 10 of the user.

The blood oxygen and electrocardiography measuring apparatus 100 may include a first clipping component 110, a second clipping component 120, a heart rate sensing pad 130, and a blood oxygen sensing component 140. The first clipping component 110 has a first pivoting portion 111 while the second clipping component 120 has a second pivoting portion 121 disposed corresponding to the first pivoting portion 111. The first clipping component 110 is pivoted with the second pivoting portion 121 of the second clipping component 120 by the first pivoting portion 111 for the first clipping component 110 to rotate with respect to the second clipping component 120 in a rotational direction R1 or in a reverse direction (i.e. a rotational direction R2). Specifically, the blood oxygen and electrocardiography measuring apparatus 100 is adapted to change between a closed state shown in FIG. 1A and an expanded state shown in FIG. 1B. When the blood oxygen and electrocardiography measuring apparatus 100 is in the expanded state shown in FIG. 1B, an angle between the first clipping component 110 and the second clipping component 120 is mainly determined by the thickness of the finger 10 of the user.

On the other hand, in order that the blood oxygen and electrocardiography measuring apparatus 100 in the expanded state shown in FIG. 1B can automatically return to the closed state shown in FIG. 1A, the blood oxygen and electrocardiography measuring apparatus 100 further includes at least one elastic member 150. The elastic member 150 is a torsion spring, for example. An end of the elastic member 150 is fixed to the first clipping component 110 while the other end thereof is fixed to the second clipping component 120. When the first clipping component 110 rotates with respect to the second clipping component 120 in the rotational direction R1 to convert the blood oxygen and electrocardiography measuring apparatus 100 to the expanded state shown in FIG. 1B, the elastic member 150 is elastically deformed and stores an elastic potential energy. When the finger 10 of the user is removed from the blood oxygen and electrocardiography measuring apparatus 100, the elastic member 150 releases the elastic potential energy to drive the first clipping component 110 to rotate with respect to the second clipping component 120 in the rotational direction R2, so as to restore the blood oxygen and electrocardiography measuring apparatus 100 to the closed state shown in FIG. 1A.

Referring to FIG. 1B, the heart rate sensing pad 130 and the blood oxygen sensing component 140 are respectively disposed on the second clipping component 120, wherein the heart rate sensing pad 130 is located between the first clipping component 110 and the second clipping component 120, and the blood oxygen sensing component 140 is located between the second clipping component 120 and the heart rate sensing pad 130. To be more detailed, the heart rate sensing pad 130 is composed of a conductive metal, for example, to be in contact with a pulp of the finger 10 of the user for measuring the heart rate of the user. In this embodiment, the heart rate sensing pad 130 may include a body portion 131, an extension portion 132 connected with the body portion 131, and an opening 133 located on the body portion 131. The blood oxygen and electrocardiography measuring apparatus 100 further includes a connection terminal 160. The connection terminal 160 is disposed in contact with the extension portion 132 to be electrically connected with the heart rate sensing pad 130. The pulp of the finger 10 of the user clipped between the first clipping component 110 and the second clipping component 120 is in contact with the body portion 131, wherein an ECG (electrocardiography) signal obtained through the body portion 131 is sequentially transmitted to the extension portion 132 and the connection terminal 160 and then transmitted to an external electronic apparatus (not shown) through an external signal line (not shown) connected on the connection terminal 160 for instantly showing the heart rate of the user through the external electronic apparatus (not shown).

The blood oxygen sensing component 140 is an optical transceiver, which is electrically connected with the extension portion 132, for example. Since the opening 133 of the heart rate sensing pad 130 exposes the blood oxygen sensing component 140, sensing light emitted by the blood oxygen sensing component 140 may pass through the finger 10 of the user through the opening 133. The sensing light includes two light beams L1 and L2 that have different wavelengths, for example. When the light beams L1 and L2 reach capillaries in the finger 10 of the user, two reflected lights L11 and L21 are generated. The reflected lights L11 and L21 pass through the opening 133 in a direction reverse to the emission direction of the sensing light (not shown) and are received by the blood oxygen sensing component 140. Then, the blood oxygen concentration of the user may be calculated by comparing the optical intensity or property of the reflected lights L11 and L21. Further, data or signals related to the blood oxygen concentration may be sequentially transmitted to the extension portion 132 and the connection terminal 160 and then transmitted to the external electronic apparatus (not shown) through the external signal line (not shown) connected on the connection terminal 160 for instantly showing the blood oxygen concentration of the user through the external electronic apparatus (not shown).

To sum up, the user clips the finger with the blood oxygen and electrocardiography measuring apparatus 100 to simultaneously measure the user's heart rate and blood oxygen concentration by the heart rate sensing pad 130 and the blood oxygen sensing component 140, or only measure the heart rate by the heart rate sensing pad 130 or only measure the blood oxygen concentration by the blood oxygen sensing component 140, so as to improve the convenience and flexibility of use of the blood oxygen and electrocardiography measuring apparatus 100.

Some other embodiments of the invention are provided as follows. It should be noted that the reference numerals and a portion of the contents in the previous embodiment are used in the following embodiment, in which identical reference numerals indicate identical or similar components, and repeated description of the same technical contents is omitted. Please refer to the description of the previous embodiment for the omitted contents, which will not be repeated hereinafter.

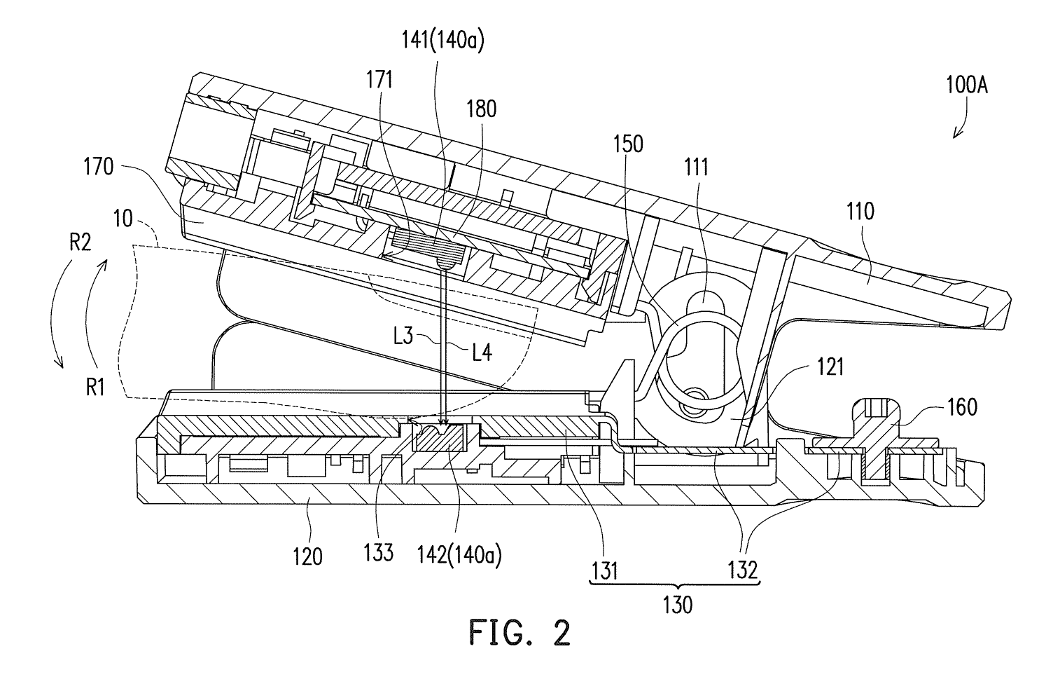

FIG. 2 is a cross-sectional schematic view depicting the blood oxygen and electrocardiography measuring apparatus in the expanded state according to another embodiment of the invention. Referring to FIG. 2, a blood oxygen and electrocardiography measuring apparatus 100A of FIG. 2 is similar to the blood oxygen and electrocardiography measuring apparatus 100 of the previous embodiment, and a difference lies in that: the blood oxygen and electrocardiography measuring apparatus 100A further includes a finger pad 170 disposed on the first clipping component 110, an optical transmitter 141 disposed on the first clipping component 110, and an optical receiver 142 disposed on the second clipping component 120 corresponding to the optical transmitter 141.

Specifically, the finger pad 170 is located between the first clipping component 170 and the second clipping component 120 and has an opening 171 disposed corresponding to the optical transmitter 141. The finger pad 170 may be composed of a conductive metal or an insulating material. The optical transmitter 141 and the optical receiver 142 constitute a blood oxygen sensing module 140a of this embodiment. The optical transmitter 141 is located between the first clipping component 170 and the finger pad 170 and is exposed by the opening 171 of the finger pad 170. The optical receiver 142 is located between the second clipping component 120 and the heart rate sensing pad 130 and is exposed by the opening 133 of the heart rate sensing pad 130. Moreover, the blood oxygen and electrocardiography measuring apparatus 100A further includes a circuit board 180 disposed between the first clipping component 110 and the finger pad 170. The optical transmitter 141 is electrically connected with the circuit board 180, such that a control unit (not shown) may control emission of a sensing light of the optical transmitter 141 through the circuit board 180. The optical receiver 142 is electrically connected with the extension portion 132.

Since the opening 171 of the finger pad 170 exposes the optical transmitter 141, the sensing light emitted by the optical transmitter 141 may pass through the finger 10 of the user through the opening 171. The sensing light includes two light beams L3 and L4 that have different wavelengths, for example. After the light beams L3 and L4 pass through capillaries in the finger 10 of the user, the light beams L3 and L4 travel along the emission direction of the sensing light and pass through the opening 133 corresponding to the opening 171 to be received by the optical receiver 142. Then, the blood oxygen concentration of the user may be calculated by comparing the optical intensity or property of the light beams L3 and L4. Further, data or signals related to the blood oxygen concentration may be sequentially transmitted to the extension portion 132 and the connection terminal 160 and then transmitted to the external electronic apparatus (not shown) through the external signal line (not shown) connected on the connection terminal 160 for instantly showing the blood oxygen concentration of the user through the external electronic apparatus (not shown).

In conclusion, the blood oxygen and electrocardiography measuring apparatus of the invention is a finger clip type blood oxygen and electrocardiography measuring probe, for example. When the user's finger is clipped between the first clipping component and the second clipping component, the heart rate sensing pad is in contact with the user's finger to measure the heart rate of the user and the blood oxygen sensing component (or blood oxygen sensing module) measures the blood oxygen concentration of the user through a non-contact optical sensing mode. In other words, simply by clipping the finger between the first clipping component and the second clipping component, the user is able to simultaneously measure the heart rate and blood oxygen concentration by the heart rate sensing pad and the blood oxygen sensing component, or only measure the heart rate by the heart rate sensing pad or only measure the blood oxygen concentration by the blood oxygen sensing component, so as to improve the convenience and flexibility of use of the blood oxygen and electrocardiography measuring apparatus.

It will be apparent to those skilled in the art that various modifications and variations can be made to the disclosed embodiments without departing from the scope or spirit of this invention. In view of the foregoing, it is intended that the invention covers modifications and variations provided that they fall within the scope of the following claims and their equivalents.

What is claimed is:

1. A blood oxygen and electrocardiography measuring apparatus, comprising:
a first clipping component;
a second clipping component pivoted with the first clipping component;
a heart rate sensing pad disposed on the second clipping component and located between the first clipping component and the second clipping component; and
a blood oxygen sensing component disposed on the second clipping component and located between the second clipping component and the heart rate sensing pad, wherein the blood oxygen sensing component is exposed from the heart rate sensing pad.

2. The blood oxygen and electrocardiography measuring apparatus according to claim 1, wherein the blood oxygen sensing component is an optical transceiver.

3. The blood oxygen and electrocardiography measuring apparatus according to claim 1, wherein the heart rate sensing pad comprises a body portion, an extension portion connected with the body portion, and an opening located on the body portion, wherein the blood oxygen sensing component is electrically connected with the extension portion and the opening exposes the blood oxygen sensing component.

4. The blood oxygen and electrocardiography measuring apparatus according to claim 3, further comprising:
a connection terminal disposed in contact with the extension portion to be electrically connected with the heart rate sensing pad.

5. The blood oxygen and electrocardiography measuring apparatus according to claim 1, further comprising:
at least one elastic member connecting the first clipping component and the second clipping component.

6. A blood oxygen and electrocardiography measuring apparatus, comprising:
a first clipping component;
a second clipping component pivoted with the first clipping component;
a finger pad disposed on the first clipping component and located between the first clipping component and the second clipping component;
a heart rate sensing pad disposed on the second clipping component and located between the first clipping component and the second clipping component; and
a blood oxygen sensing module, comprising:
an optical transmitter disposed on the first clipping component and located between the first clipping component and the finger pad, wherein the optical transmitter is exposed from the finger pad; and
an optical receiver disposed on the second clipping component corresponding to the optical transmitter and located between the second clipping component and the heart rate sensing pad, wherein the optical receiver is exposed from the heart rate sensing pad.

7. The blood oxygen and electrocardiography measuring apparatus according to claim 6, wherein the heart rate sensing pad comprises a body portion, an extension portion connected with the body portion, and an opening located on the body portion, wherein the optical receiver is electrically connected with the extension portion and the opening exposes the optical receiver.

8. The blood oxygen and electrocardiography measuring apparatus according to claim 7, further comprising:
a connection terminal disposed in contact with the extension portion to be electrically connected with the heart rate sensing pad.

9. The blood oxygen and electrocardiography measuring apparatus according to claim 6, further comprising:
at least one elastic member connecting the first clipping component and the second clipping component.

10. The blood oxygen and electrocardiography measuring apparatus according to claim 6, further comprising:
a circuit board disposed between the first clipping component and the finger pad, wherein the optical transmitter is electrically connected with the circuit board.
